(19) United States
(12) Patent Application Publication
(10) Pub. No.: US 2014/0141385 A1
(43) Pub. Date: May 22, 2014

(54) INTEGRATIVE SYSTEM FOR DENTAL PROCEDURES

(71) Applicant: Abraham Taub, Rishon Lezion (IL)

(72) Inventor: Abraham Taub, Rishon Lezion (IL)

(73) Assignee: CREATIVE TEAM INSTRUMENTS LTD, Rishon Le Zion (IL)

(21) Appl. No.: 14/125,518

(22) PCT Filed: Jul. 3, 2012
(86) PCT No.: PCT/IL2012/000270

§ 371 Date: Dec. 11, 2013

(30) Foreign Application Priority Data
Jul. 24, 2011 (IL) 214265

(51) Int. Cl. A61B 5/053; A61C 19/04; A61B 5/00; A61C 1/08; A61B 6/03; A61B 1/24; A61B 1/04; A61B 5/06; A61C 8/00; A61B 6/14

(52) U.S. Cl. CPC A61B 5/0534; A61C 8/0089; A61C 19/04; A61B 5/742; A61B 6/14; A61B 6/032; A61B 1/24; A61B 1/04; A61B 5/066; A61B 5/0002; A61C 1/084; A61B 5/7405; A61B 5/7455; A61B 5/72
USPC 433/27; 433/173

(57) ABSTRACT

A dental system including a computer and a display, and one or more dental sensors communicating with the computer. Some of the dental sensors could be operative to provide information or an image of one or more oral components and communicate the image to the computer or computer memory. The image stored in the memory could be a graphic image and the information could also include an image and a predetermined dental treatment protocol. It could be an X-ray image, a two-dimensional image, a three-dimensional image, a panoramic image, and a CT image. This superimposed dental image, received from a number of dental sensors, and the dental instrument image facilitates correction of errors and inaccuracies in instrument location and/or angle of dental instrument positioning and tooth penetration as well as various measurements such as number and length of root canals and other parameters required for performing an accurate dental procedure.

### TECHNICAL FIELD

The current method and apparatus relate to systems for dental procedures and in particular to systems capable of handling a plurality of dental procedures.

### BACKGROUND

Contemporary dentistry is aimed to maintain oral health as well as improve the aesthetic appearance of the mouth and involves periodontal (around the tooth), endodontic (inside the tooth) and orthodontic (preventing and correcting irregularities of the teeth) procedures that involve the use of a variety of dental tools and instruments as well as accessories that provide pre-treatment and intra-treatment information to the dentist regarding parameters such as the location at which the treatment is being performed and the particular tooth roots number and their length.

Currently, such information is obtained from several sources, each requiring close analysis and processing by the dentist. Some of the information analysis and processing is done prior to the procedure in order to plan the procedure steps, while oftentimes additional information needs to be acquired in real time, requiring stopping the treatment, obtaining the additional information such as, for example, X-ray or other images, analyzing and processing this information and carrying on with the procedure. This may occur one or more times throughout the procedure causing the treatment to become lengthy and tiring to the patient as well as costly to the dentist.

Moreover, the current information obtaining systems require the dentist to move away from the patient such as when X-raying the patient or evaluating the acquired information, e.g., viewing X-ray films.

In one example a root canal procedure can include the following steps:
Obtaining an image of the affected tooth employing an X-ray film or digital image.
Analyzing and processing the image and making a decision whether to fill the tooth (with or without root canal treatment) or extract the tooth.
In case of a root canal treatment, analyzing the same or an additional X-ray image to assess the number of canals in the affected tooth.
Opening the pulp chamber of the affected tooth and locating the entries into all of the existing root canals employing an ordinary dental mirror, an intra-oral camera, or an endoscope.
Assessing the length of each of the canals employing, for example, an dental impedance meter.
Treating each of the existing canals to its full previously measured working length; and
Finally, filling the affected tooth.

In another example, a tooth implantation procedure aimed, for example, to improve the aesthetic appearance of the mouth and restoration of mastication function can include the following steps:
Obtaining a panoramic image of the patient's jaws (X-ray film or digital image).
Obtaining Computerized Tomography (CT) images of the patient's jaws.
Carefully analyzing the obtained panoramic and CT images and making a decision regarding the number of implants to be inserted, their exact location and the direction or angle at which each of the implants should be inserted.
Locating and identifying (i.e., marking) each of the selected implantation points in the patient's jaw.
Placing the drill exactly at the selected and identified points and orienting the drill in an assessed optimal direction based on the previously analyzed images and then drilling the hole.
Inserting the implant.

Many of the above described steps leave room for error and inaccuracies in instrument location and/or angle of instrument positioning and tooth penetration as well as various measurements such as number and length of root canals and other parameters that are required for performing a time-efficient accurate dental procedure.

### SUMMARY

Presented is a dental system that includes a computer, a display, and one or more dental sensors communicating with the computer. Some of the dental sensors could be operative to provide an image and communicate it to the computer or computer memory. The image or information could be such as an image of one or more oral components. The image stored in the memory could be a graphic image, digital values related to an oral component status, and the information could also include an image and a predetermined dental treatment protocol. It could be an X-ray image, a two-dimensional image, a three-dimensional image, a panoramic image, and a CT image.

The dental sensor could be such as a Digital Dental X-ray sensor, an X-ray film scanner, an intraoral camera, a dental impedance probe and a spatial orientation sensor. The system could also include one or more dental instruments.

The dental system could handle information and images received from one or more of dental sensors and superimpose this information into one graphic image providing the dentist with real time information on progress of different dental procedures and guiding the dentist in applying or using different dental tools. The superimposed image could include information received from the at least one dental sensor and/or stored in the memory and the orientation and location of the dental instrument relative to a displayed image.

This superimposed dental image, received from a number of dental sensors, and the dental instrument image facilitates correction of errors and inaccuracies in instrument location and/or angle of instrument positioning and tooth penetration as well as various measurements such as number and length of root canals and other parameters that are required for performing a time-efficient accurate dental procedure.

The computer, actually the processor, facilitates integration of information received from the dental sensor or stored in the memory with information received from the dental instrument. The integrated oral component and dental instrument images include the dental instrument spatial orientation and location relative to the oral component and could provide in real time an output regarding an optimal position and orientation of the dental instrument.

The communication between the computer and dental sensors and dental instruments could be via a wired or via a wireless interface. The dental system display could be a chair-side display allowing a dentist to watch the display and operate the dental instrument concurrently.

### BRIEF DESCRIPTION OF THE DRAWINGS

The present apparatus and method will be understood and appreciated more fully from the following detailed description, taken in conjunction with the drawings in which:

FIG. 1 is a block diagram of an example of a system for handling a plurality of dental procedures;

FIG. 2 is a simplified illustration of an example of a dental impedance probe used in the system of FIG. 1;

FIG. 3 is an a simplified illustration of an example of a dental impedance probe used in the system of FIG. 1;

FIG. 4 is a flow chart diagram of an example of the interaction between components of a system for handling a plurality of dental procedures;

FIG. 5 is a flow chart diagram of another example of the interaction between components of a system for handling a plurality of dental procedures; and

FIG. 6 is a flow chart diagram of another example of the interaction between components of a system for handling a plurality of dental procedures.

### DETAILED DESCRIPTION

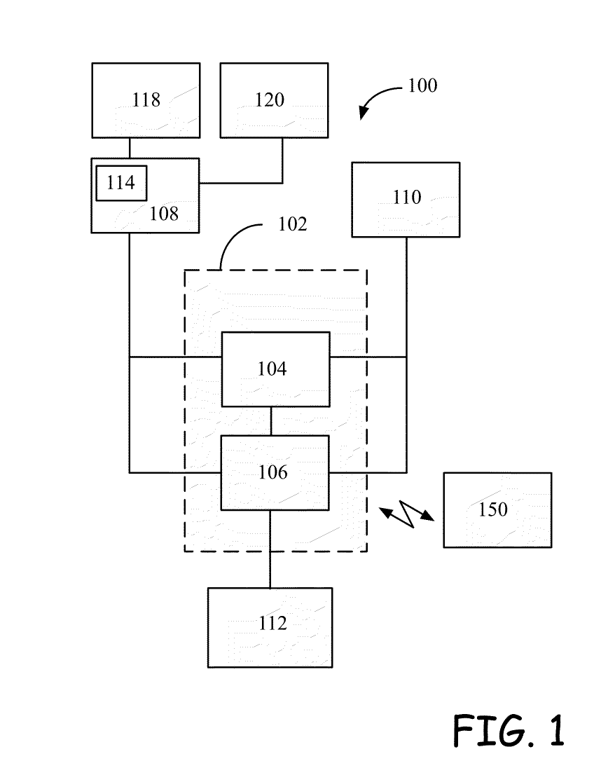

Referring now to FIG. 1, which is a block diagram of an example of an integrative system for dental health and dental aesthetic procedures. An integrative dental system 100 can include system components such as a computer 102, which could be a personal computer (PC) or a portable computer such as a laptop computer or tablet computer or any other suitable computer, with a memory 104 and a processor 106 communicating with each other and with one or more dental instruments 108, one or more dental sensors 110 and one or more display units 112 that could be located patient chair-side. Additionally and optionally, computer 102 could also communicate with a remote computer 150.

Dental instrument 108 can also include one or more dental sensors 114 and can also include a harness 116 accommodating electrical and mechanical power cables supplying dental instrument 108 with electrical and/or mechanical power from respective electrical 118 and mechanical 120 (e.g., a motor, a vibrator) sources, as well as fluid supply and drainage tubes.

Dental sensor 110 could be one or more of a digital dental X-ray sensor, an X-ray film scanner, an intra-oral camera, an endoscope such as, for example, an intraoral mini USB endoscope, a spatial orientation sensor, an implant locator such as that described in the Patent Cooperation Treaty Publication (PCT) WO2011/064768 to the same assignee, a thermal image sensor, a dental impedance sensor and any other similar sensor capable of providing image, graphic or any other information regarding at least one oral component, such as a tooth, a dental implant, mandibular or maxillary bone, gingiva (i.e., gums), tongue or similar.

Alternatively or additionally relevant information can be obtained in digital form from a standalone computerized tomography imaging unit, a magnetic resonance imaging unit (MRI) or an X-ray unit. The information can be obtained via standard communication links or using portable storage media devices. Information can also be obtained from conventional image capturing devices such as a film or digital camera, picture prints or X-ray films, be digitalized by scanning and input into system 100 memory 104.

Dental sensor 114 can be one or more of an endoscope, a dental impedance probe, an implant locator such as that described in the PCT Publication WO2011/064768 to the same assignee, and include one or more a spatial orientation sensor, a thermal image sensor, an impedance sensor and any other similar sensor capable of providing graphic or any other information regarding at least one oral component, such as a tooth, mandibular or maxillary bone, gingiva (i.e., gums), tongue or similar.

A number of dental sensors could be used concurrently. For example, dental sensor 110 could be an intraoral mini USB endoscope such as USBCam commercially available from Schick Technologies, Inc., Long Island City, NY 11101 U.S.A., or another similar endoscope, and dental sensor 114 could be a dental impedance probe or measurement device.

The spatial orientation sensor could be any one of a group of 3-axis angular rate gyroscope and a 3-axis accelerometer, which determine two fixed vectors in space. The two fixed vectors, as disclosed in the Patent Cooperation Treaty Publication WO2011/089606, determine a geometrical plane whose normal specifies a unique orientation.

Information from dental sensors 110 and 114 can be communicated to the computer 102 via a wired or wireless communication links, processed in processor 106 and/or stored in memory 104 for future use. Information processed by processor 106 or stored in memory 104 can be displayed on display unit 112.

Memory 104 can be any storage device such as a hard disk, disk-on-key, compact disc (CD) flash card memory or internal random access memory configured to store graphic information such as, for example, CT-scan, X-ray or MRI images.

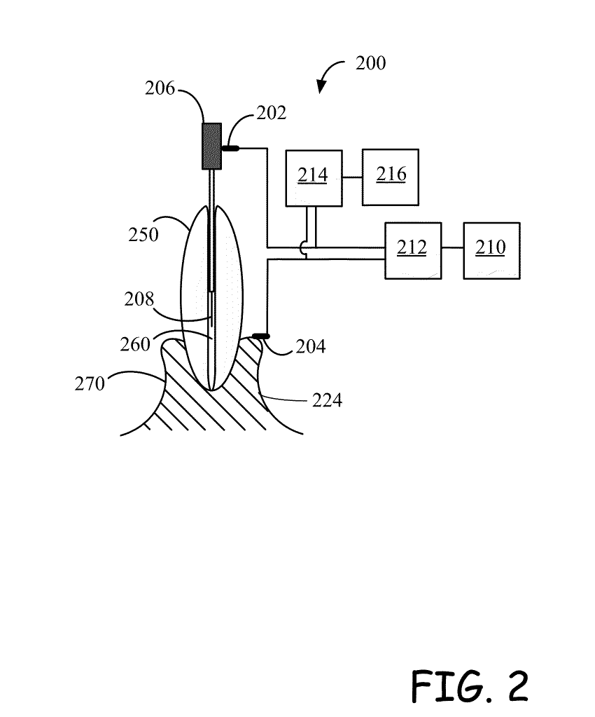

As shown in FIG. 2, integrative dental system 100 can include a dental impedance probe 200 including two or more electrode terminals 202 and 204 configured to form a contact with a segment of a patient body, which could be a gingival tissue or a lip, and a conductive probe 206 having a probe tip 208 capable of being inserted into tooth 250 root canal 260. Terminal 202 can be connected to probe 206 tip 208 whereas terminal 204 can be connected to the patient's to gingival tissue 270 or the lip (not shown).

An AC current generator 210 operative to provide an AC test current signal Ig at one or more frequencies (f) between 100 Hz and 100 KHz, can be connected to terminals 202 and 204 via an AC current driver interface 212. An analog front end unit 214 facilitates measurement of an AC voltage Vi(f) caused by the AC test current signal Ig between electrodes 202 and 204 across the root canal chamber 260 impedance Zr. Typically, the voltage Vi(f) could be a vector of a certain length oriented at an angle to the current Ig. The voltage could be expressed as Vi(f)=Ig*|Zr(f)⊕*e^{je}. Angle Ø(f) between the Real and the Imaginary components of voltage Vi(f) can be measured as well. An electronic controller 216 is operative to convert the measured by analog front end unit 214 the voltage Vi(f) and Ø(f) analog values to a digital data and transmit the obtained results to processor 106 for further interpretation and graphical presentation by display unit 112 in real time (FIG. 1). The measured values of Vi(f) and Ø(f) could be processed to indicate on that the apex of a particular root channel has been reached or on presence of additional to the main root side root channels.

Dental implant prosthetic procedures exist for a long period. Prior to conducting further prosthetic work the dentist has to be sure that the dental implant has been mechanically stable and biologically integrated into the jaw bone. (The term “dental implant” as used in the present disclosure includes implant fixture and implant post.) Currently existing diagnostic tools, assisting the dentist in establishing that the dental implant has been mechanically stable and biologically integrated into the jaw bone or what is termed successful osseointegration, can be used only after dissecting the gingiva, opening the implant and attaching a special insert to it. Such method is not applicable for implants located under the gingiva. Although the osseointegration process is highly individual, but to be on the sure side the dentists prefer to wait three-six months and only after this period to perform a cut in the gingival tissue accessing the dental implant and mechanically checking the dental implant stability.

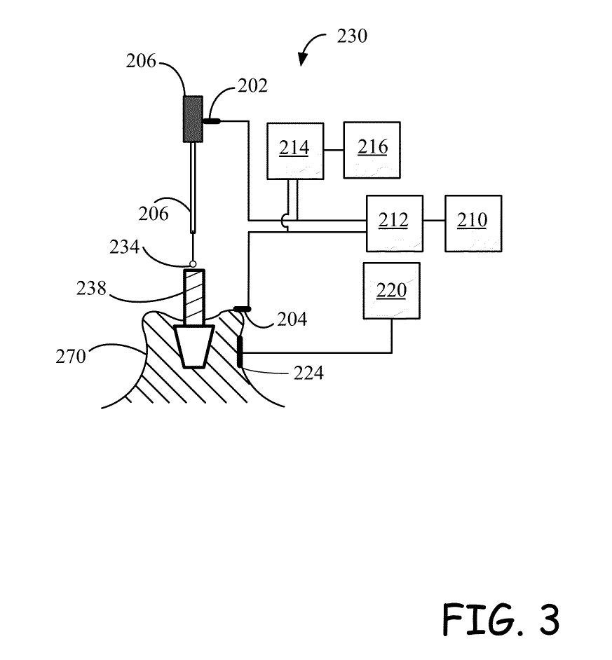

The present integrated dental system offers a solution to this problem. FIG. 3 is a simplified illustration of an example of a dental impedance probe used in the system of FIG. 1. Integrative dental system 100 can include a dental impedance probe 300 including two or more electrode terminals 202 and 204 configured to form a contact with a segment of a patient body, and a conductive probe 306 having a probe tip 310, terminated by a sharp termination adapted to penetrate gingival tissue 270 and facilitate contact with a dental implant 318. Terminal 202 can be connected to probe 300 whereas terminal 204 can be connected to the patient's gingival tissue 270 or the lip (not shown). Dental impedance probe 300 further includes a low frequency vibrator 320, mechanically connected to the probe tip 310. Vibrator 320, driven by a low frequency signal generator 324, applies the low frequency vibrations via the probe tip 310 to dental implant 318. The frequency of the vibrations could be in the range of 2 to 200 Hz. The AC test current signal (Ig) provided by AC current generator 210 of dental impedance sensor 200 typically has a frequency (f) between 100 Hz and 100 kHz and could be used to provide indication on how well the dental implant 318 has settled down or was adapted by the jaw bone 322. A low power low frequency vibrations, provided by a signal generator 324 and applied by the vibrator 320 via the tip 310 of the conductive probe 306 to the dental implant 318 would cause minute vibrations of the dental implant 318 that would modulate the impedance of the circuit between terminals 202 and 204 and respectively the amplitude of the measured AC voltage signal Vi(f) caused by the AC test current signal (Ig). In some examples the AC test signal could be a voltage signal applied to the terminals 202 and 204 via a reference resistor Rr. The amplitude of the AC voltage signal Vi(f) modulation would be inversely proportional to the level of the implant integration into the jaw bone 270 and indicate on how well dental implant 318 has settled down or was adapted by the jaw bone 322 or simply on the dental implant-jaw bone joint status. For example, large AC voltage signal amplitude changes (in excess of X%) would indicate on poor dental implant-jaw joint status. Small voltage amplitude changes (less than Y%) would indicate on good or proper dental implant-jaw bone joint status. Particular “X” and “Y” values are individual for each patient and are determined for each patient almost immediately following insertion of the implant. An implant locator such as that described in the PCT Publication WO2011/064768 to the same assignee could be used to detect location of the dental implant covered by the gingiva before applying the vibrations to the dental implant and performing AC signal amplitude modulation measurements.

In one example, AC voltage signal amplitude changes could be processed and displayed in a graphical form on display 112 (FIG. 1) superimposed with the previously measured and stored values for that particular implant, showing the tendency graph and providing the dentist with real time visual information on implant fixture jaw joint status. Display unit 112 can include a touch screen allowing the dentist to input information such as clinical findings and progress notes in real time, as well as retrieve information from memory 104. The retrieved information could be such as previous image data, for example previous panoramic views for orthodontic aesthetic procedures, treatment plans and similar information. Additionally and optionally, display unit 112 touch screen could be employed to allow the dentist to input data and employ processor 106 and memory 104 to execute necessary chair-side computing during the dental treatment procedure. Additionally and optionally, clinical findings can be recorded semi-automatic or automatically.

The communication between one or more system 100 components can be carried out via electrical and mechanical cables, USB ports and wireless communication systems such as Radio Frequency (RF), Infrared (IR) and similar.

System 100 can also include additional features such as voice and audio recording and sounding and audio and visual alarms. Dentist could record some of his/her vocal notes to be back played with the image displayed on display unit 112.

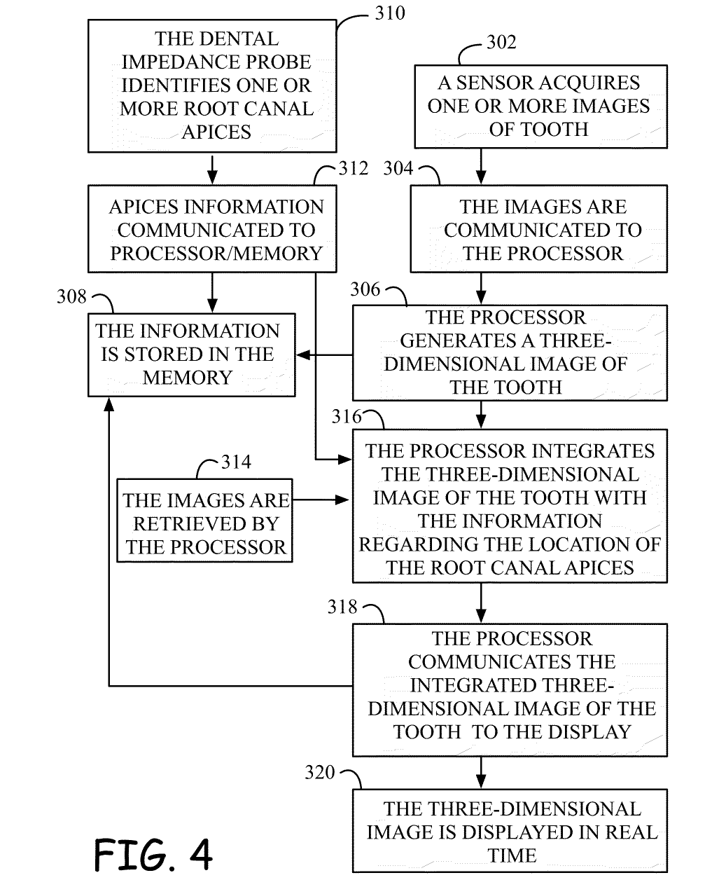

Reference is now made to FIG. 4, which is a flow chart of an example of the interaction between components of an integrative system 100 for handling a plurality of dental procedures.

As seen in block 402, a dental sensor 110 (FIG. 1) such as an X-ray image sensor or CT scanned data input can acquire in situ information such as a digital X-ray image or digital CT-scan image provided by communication or by digital portable memory of an oral component such as, for example, a tooth and communicate the image (block 404) to a processor 106 (FIG. 1). Alternatively and optionally, dental sensor 110 could be a conventional scanner and acquire an image by scanning hard copy photographs or X-ray film acquired offline and converting the images to digital images stored in memory 104. Image scanner in the capacity of a dental sensor 110 could be built-in into the enclosure of computer 102 or be a standalone image scanner.

Alternatively and optionally, the dental sensor 110 can acquire several images of an oral component taken at several angles, communicate the images to processor 106 which, in turn, can process the images to generate a three-dimensional image (block 406) of the oral component, for example, such as a tooth, a number of teeth or other oral components and store (block 408) the three-dimensional image in memory 104 (FIG. 1).

Additionally and optionally, dental sensor 110 or 114 can be a dental impedance probe, used for root canal treatment and dental implant jaw bone joint status assessment could identify presence of one or more root canal apices (block 410) and communicate root canal apices information to processor 106 (block 412) and to memory 104. Processor 106 could become operative to integrate (block 416) the three-dimensional image of the tooth with the information regarding the location of the root canal apices. The processor could further communicate the three-dimensional image of the tooth integrated with the information regarding the location of the root canal apices to a display unit 112 (block 418) and display this image (block 420) in real time, guiding the dental system operator in his work and actions.

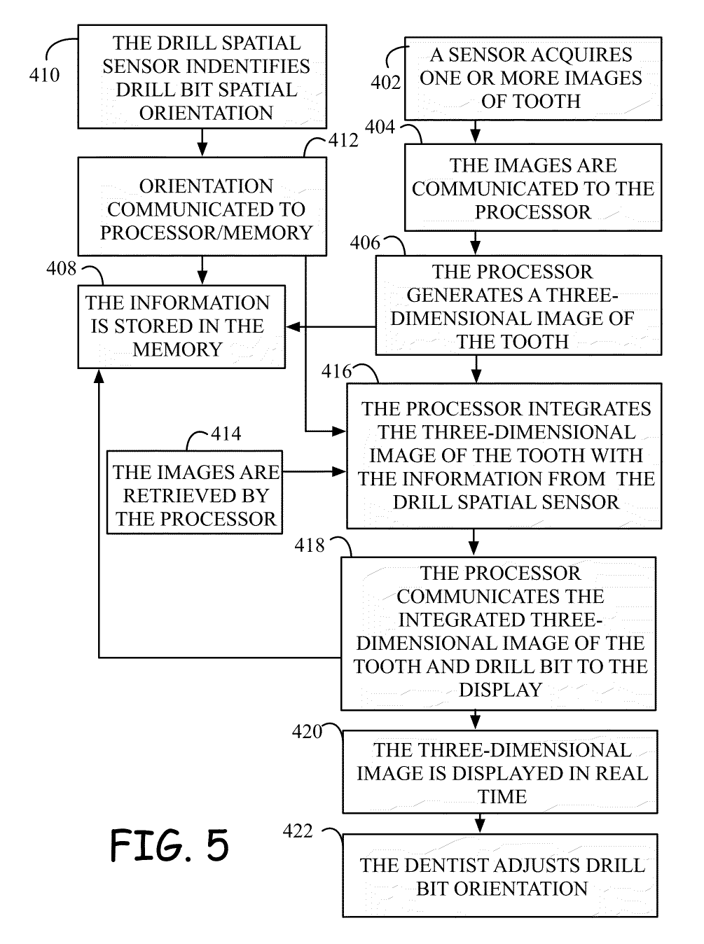

Reference is now made to FIG. 5, which is a flow chart of another example of the interaction between components of an integrative system for handling a plurality of dental procedures.

As seen in block 502, a dental sensor 110 (FIG. 1) such as an X-ray image sensor or CT-scanner digital input can acquire information regarding an oral component such as an X-ray image or CT-scan image of an oral component such as, for example, a tooth or other oral component and communicate the image (block 504) to a processor 106 (FIG. 1). Alternatively and optionally, the dental sensor 110 can acquire several images of an oral component taken at several angles, communicate the images to processor 106 which, in turn, can process the images to generate a three-dimensional image (block 506) of the oral component and store (block 508) the three-dimensional image in memory 104 (FIG. 1).

Alternatively or additionally the relevant information can be obtained in digital form via regular communication links from standalone computerized tomography imaging unit, a magnetic resonance imaging unit (MRI), an X-ray unit.

Additionally and optionally, an instrument 108 (FIG. 1) can be a drill having a drill bit and including a spatial sensor 114 (FIG. 1), which could be a 3-axis accelerometer coupled with a 3-axis gyro supporting identification (block 510) of the spatial orientation of the drill bit and comparing the current drill bit orientation with the previously measured reference drill bit spatial orientation stored in memory 104 (FIG. 1). Processor 106 can retrieve the reference drill bit spatial orientation from the memory (block 514) and integrate in real time the current drill bit spatial orientation with (block 516) the three-dimensional image of the jaw when drilling in the jaw, e.g., when preparing the jaw bone for a dental implant insertion. The integrated in real time drill bit spatial orientation with the reference spatial orientation could be communicated (block 518) to display 112 (FIG. 1) The display can display the integrated image (block 520) to the dentist in real time so the dentist can make any required adjustments (block 522) of the drill bit orientation and achieving optimal positioning and orientation prior to activating the drill.

Display 112 could be placed chair-side so that to allow the dentist to watch the display and real time drill image or drilling process progress displayed thereon and concurrently operate the dental instrument such as adjusting the spatial orientation of the drill bit.

Alternatively and optionally, the spatial identification of the drill bit orientation (block 510) can be continuous so that the dentist can continuously adjust (block 522) the location and orientation of the drill bit or any other dental instrument 108 throughout the procedure.

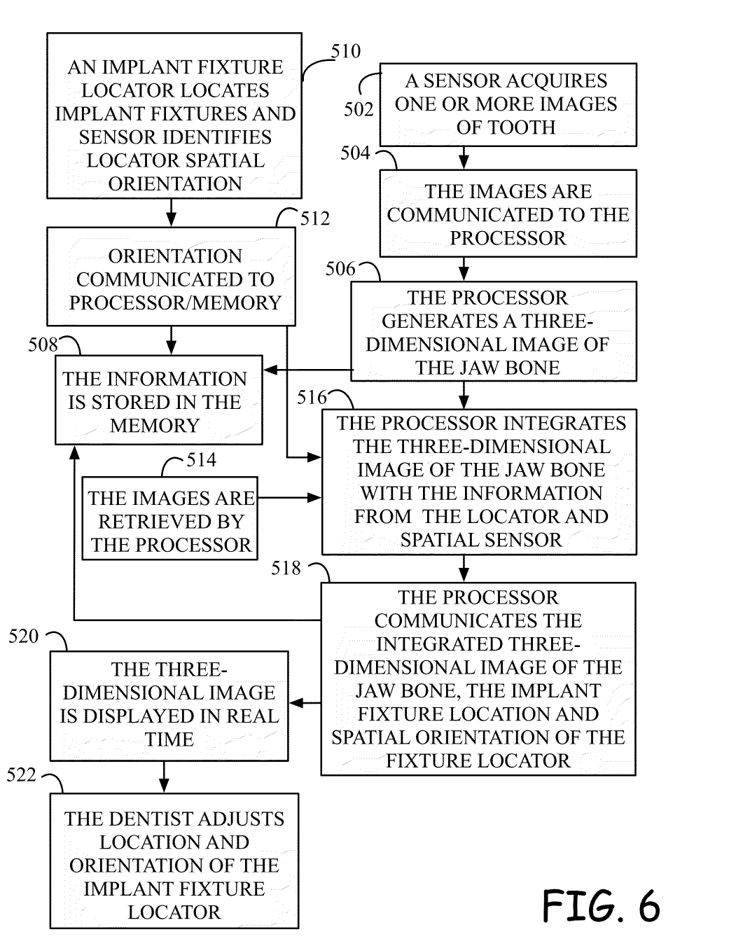

In another example and as shown in FIG. 6, which is a flow chart of another example of the interaction between components of an integrative system for dental procedures, a dental sensor 110 (FIG. 1) such as an X-ray unit sensor or CT-scanner digital input can acquire (block 602) information regarding an oral component such as an X-ray image or CT-scan image of an oral component such as, for example, a jaw bone and communicate the image (block 604) to a processor 106 (FIG. 1). Alternatively and optionally, the dental sensor 110 can acquire several images of an oral component taken at several angles, communicate the images to processor 106 which, in turn, can process the images to generate a three-dimensional image (block 606) of the oral component and store (block 608) the three-dimensional image in memory 104 (FIG. 1).

Additionally and optionally, an instrument 108 (FIG. 1) can be an implant fixture locator facilitating localization and identification of buried in the gum/jaw dental fixture implants and include a spatial sensor 114 (FIG. 1) supporting identification of the spatial orientation of locator 108 (block 610). Implant fixture locator 108 can communicate to processor 106 (FIG. 1) and/or store (block 608) in memory 104 (FIG. 1) information regarding the identified location of implant fixtures while dental sensor 114 can communicate information regarding the spatial orientation of fixture implant locator 108 (block 612). Processor 106 can retrieve from the memory implant fixture spatial location images (block 614) and integrate (block 616) the three-dimensional image of the jaw bone or other oral component stored in memory 104 with the location of the dental implants received in real time from the implant fixture locator 108 and the implant fixture locator spatial orientation received from dental sensor 114 (FIG. 1). Processor 106 could communicate the integrated three-dimensional image of the jaw-bone, the implant fixture location, and spatial orientation of the fixture locator (block 618) to display 112 (FIG. 1). The display can display the integrated three-dimensional image of the imaged jaw bone including the location of identified implant fixtures and spatial orientation of implant fixture locator 108 relative to the three-dimensional image (block 620) to the dentist in real time so the dentist can make any required adjustments (block 622) of the location and orientation of the implant fixture locator achieving optimal localization of the implant fixture.

It will be appreciated by persons skilled in the art that the present system and methods is not limited to what has been particularly shown and described hereinabove. Rather, the scope of the invention includes both combinations and sub-combinations of various features described hereinabove as well as modifications and variations thereof which would occur to a person skilled in the art upon reading the foregoing description and which are not in the prior art.

1-31. (canceled)

32. A dental system comprising:
a dental impedance probe comprising two or more electrode terminals and a conductive probe terminated by a sharp tip adapted to penetrate gingival tissue and facilitate contact with a dental implant;
a vibrator mechanically connected to the conductive probe and driven by a low frequency signal generator, the vibrator applies low frequency vibrations via the conductive probe tip to the dental implant to cause minute vibrations of the dental implant that would modulate the impedance of the circuit between the two or more electrode terminals and respectively amplitude of the measured AC voltage signal caused by an AC test current signal between the dental implant and a jaw bone; and
wherein amplitude modulation of the measured AC voltage signal caused by the AC test current signal is indicative of dental implant jaw bone joint status.

33. The dental system according to claim 32, wherein amplitude modulation of the measured AC voltage signal is inversely proportional to level of the dental implant integration with the jaw bone and indicates on how well an implant was adapted by the jaw bone.

34. The dental system according to claim 32, wherein the vibrator vibrates at a frequency in the range of 2 to 200 Hz.

35. The dental system according to claim 32, wherein the AC test current signal has a frequency between 100 Hz and 100 kHz.

36. A method for detecting a dental implant status, the method comprising:
providing two electrodes and bringing a first electrode into contact with a dental implant and a second electrode into contact with a patient's body;
providing an AC test current signal having one or more frequencies between the first and the second electrode and measuring an AC voltage signal amplitude caused by the AC test current signal across the first and second electrodes;
applying low frequency vibrations to the dental implant and measuring amplitude modulation of the AC voltage signal amplitude across the first and second electrodes; and
wherein the AC voltage signal amplitude changing across the first and second electrodes indicates a status of the dental implant and jaw bone joint.

37. A dental system comprising:
a computer having a memory and communicating with at least one dental sensor capable of at least providing an image;
at least one dental instrument; and
at least one display; and
wherein the computer is capable of processing information received from the at least one dental sensor and/or stored in the memory and from the dental instrument and display in real time at least one of an orientation and location of the dental instrument relative to the image provided by the dental sensor.

38. The dental system according to claim 37, wherein the image provided by the dental sensor comprises at least one oral component.

39. The dental system according to claim 37, wherein the image stored in the memory comprises at least one of an X-ray image, a two-dimensional image, a three-dimensional image, a panoramic image, and a CT image.

40. The dental system according to claim 37, wherein the dental sensor is at least one of a group of dental sensors consisting of a Digital Dental X-ray sensor, an X-ray film scanner, an intraoral camera, an dental impedance probe and a spatial orientation sensor.

41. The dental system according to claim 37, wherein the dental sensor is at least providing a signal being converted into an image.

42. The dental system according to claim 37, wherein the orientation of the dental instrument is displayed superimposed in real time with the image stored in the memory or with an image provided by the dental sensor.

43. The dental system according to claim 37, wherein the computer is further capable of integrating information received from the dental sensor or stored in the memory with information received from the dental instrument regarding at least one of the dental instrument spatial orientation and location relative to an oral component and providing in real time an output regarding an optimal position and orientation of the dental instrument.

44. The dental system according to claim 37, wherein the computer also comprises a processor capable of super imposing in real time an image of the dental instrument location and/or orientation on the image acquired by the dental sensor into an integrated common three-dimensional image and communicating the integrated common image to the display.

45. The dental system according to 37, wherein the image stored in the memory is at least one of a graphic image and a predetermined dental treatment protocol.

46. The dental system according to 37, wherein an integrated image and the output regarding optimal position and orientation of the dental instrument provides a dental system operator with real time feedback for correct tool positioning and orientation.

47. The dental system according to 37, wherein the output regarding the optimal position and orientation of the dental instrument is provided in at least one form consisting of a graphic image display, an auditory signal, an optical signal and a mechanical vibration signal.

48. The dental system according to claim 37, wherein the communication is at least one of a group of communications consisting of communication via a physical wire or via a wireless connection.

49. The dental system according to claim 37, wherein the display is a chair-side display so that to allow a dental system operator to watch the display and operate the dental instrument concurrently.
